(19) United States
(12) Patent Application Publication
(10) Pub. No.: US 2003/0153937 A1
(43) Pub. Date: Aug. 14, 2003

(54) Apparatus for use in the treatment of choanal atresia

(76) Inventors: Ali Saeed Mohammed Al-Qahtani, Wadi Al-Dawasir (SA); Farouk Mahmoud Ahmed Messahel, Wadi Al-Dawasir (SA)

Correspondence Address: BACON & THOMAS, PLLC, 625 SLATERS LANE, FOURTH FLOOR, ALEXANDRIA, VA, 22314

(21) Appl. No.: 10/359,253

(22) Filed: Feb. 6, 2003

(30) Foreign Application Priority Data
Feb. 7, 2002 (GB) 0202800.9

(51) Int. Cl. A61F 11/00

(52) U.S. Cl. 606/162

(57) ABSTRACT

Apparatus for use in the treatment of choanal atresia and in the prevention of stenosis following choanal atresia repair in infants comprises two choanal tubes adapted to lie side-by-side each of a plastics material approved for use in humans and each having a lengthwise extending flexible metallic insert embedded internally within its wall(s). The ends of a strip of plastics material are attached one to one end of each of the choanal tubes and a bridge of plastics material extends between and is removably attached to the choanal tubes at or towards their ends remote from the strip of plastics material. The arrangement is such that, in use, the choanal tubes are positioned in the nasal passageways and transnasal openings of an infant with the plastics strip extending behind the cartilage which separates the nasal passageways, and with the bridge positioned adjacent the columella of the infant.

This invention relates to apparatus for use in the treatment of choanal atresia, especially for the prevention of stenosis following choanal atresia repair.

Choanal atresia (obstruction of the posterior nasal openings) is the most common congenital nasal anomaly of newborn infants with an incident of approximately 1 in 5000 to 7000 live births which necessitates urgent medical intervention. A newly born infant is obligatory a nose breather; mouth-breathing is a learned reflex that can take from hours to days to acquire. Until opening of the mouth is learned, cyclical respiratory obstructions can occur, As a child falls asleep, its mouth closes, and a progressive obstruction can result, starting with a stridor and progressing to increased effort and cyanosis. Normally the initial alerting event is the first feeding of a newborn infant. As the child startles, a progressive obstruction of the airway occurs with cyanosis and hypoxia to the point of aspiration of milk and choking. Accordingly a newborn infant can fail to thrive.

This congenital condition is normally treated by surgical opening of the choanae using a drill, an endoscope or a laser.

Whereas such treatments provide immediate relief, surgically formed openings tend to re-close. To this extent ear, nose and throat (ENT) surgeons differ widely on how to prevent restenosis of the formed openings. Some surgeons place stents fashioned from several hard and soft materials in the formed openings. Most common of these is a stent made by modifying an endotacheal tube. Such a stent is disclosed in UK-A-2352976. Other surgeons prefer to perform serial dilatation of the openings once a week for 4-6 weeks. Still others maintain patency by regular bouginage every two months. All of these procedures are time consuming and none is entirely satisfactory.

The Applicants believe that a cause of failure of stents produced by modifying endotracheal tubes is related to the material from which such stents are produced. Tracheal tubes are generally produced from polyvinylchloride which softens at body temperature and can consequently collapse under an attempt at re-epithelisation of choanae. In addition, repeated anaesthetics unnecessarily subjects newborn infants to the hazards of multiple anaesthetics and tracheal intubation. Also, stents made of rubber may induce localised toxic tissue reaction.

One object of this invention is to provide apparatus for use in the treatment of choanal atresia which does not suffer from, or at least alleviates, the disadvantages discussed above.

According to the present invention, there is provided apparatus for use in the prevention of stenosis following choanal atresia repair in infants, the apparatus comprising two choanal tubes adapted to lie side-by-side each of a plastics material approved for use in humans and each having a lengthwise extending flexible metallic insert embedded internally within its wall(s), a strip of plastics material whose ends are attached one to one end of each of the choanal tubes, and a bridge of plastics material extending between and removably attached to the choanal tubes at or towards their ends remote from the strip of plastics material, the arrangement being such that, in use, the choanal tubes are positioned in the nasal passageways and transnasal openings of an infant with the plastics strip extending behind the cartilage which separates the nasal passageways, and with the bridge positioned adjacent the columella of the infant.

Each metallic insert may comprise a spiral of metallic material embedded within the respective choanal tube. The metallic material may be stainless steel. In a preferred embodiment of the invention, each metallic insert comprises a stainless steel wire coil embedded internally within the respective choanal tube.

Each choanal tube may be produced from silicone rubber or polyvinylchloride.

Each metallic insert may extend over the major part of the length of the respective choanal tube.

The ends of the plastics strip may be permanently or releasably attached to the ends of the choanal tubes.

The bridge may be held in place by a thread which passes through holes formed in the choanal tubes.

A piece of resilient material may be attached to that side of the bridge opposite the plastics. strip. The resilient material may be produced from a sponge-like material.

The diameter of each choanal tube preferably lies in the range 2.5 to 4.0 mm.

In another aspect, the invention provides apparatus for use in the treatment of choanal atresia which comprises two side-by-side choanal tubes each of a material approved for use in humans and each reinforced with an embedded lengthwise extending filament coil, and a strip of material approved for use in humans whose ends are attached to one end of each tube.

The material of the choanal tubes and the strip may be plastics, for example polyvinylchloride.

A bridge of plastics material may be releasably secured to each choanal tube at positions at or towards the ends of the tubes remote from the strip of plastics material.

The embedded filament coil may comprise a spiral of metallic material, for example stainless steel.

The plastics strip may be permanently or separably attached to the ends of the two choanal tubes.

A specific embodiment of the invention will now be described by way of example with reference to the accompanying diagrammatic drawings in which:

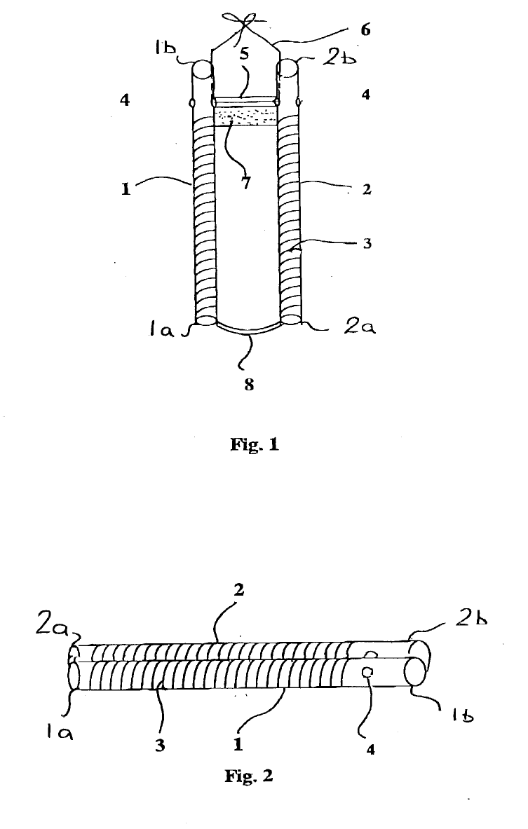

FIG. 1 is a perspective view of apparatus for use in the treatment of choanal atresia in accordance with the invention;

FIG. 2 is a side view of the apparatus shown in FIG. 1;

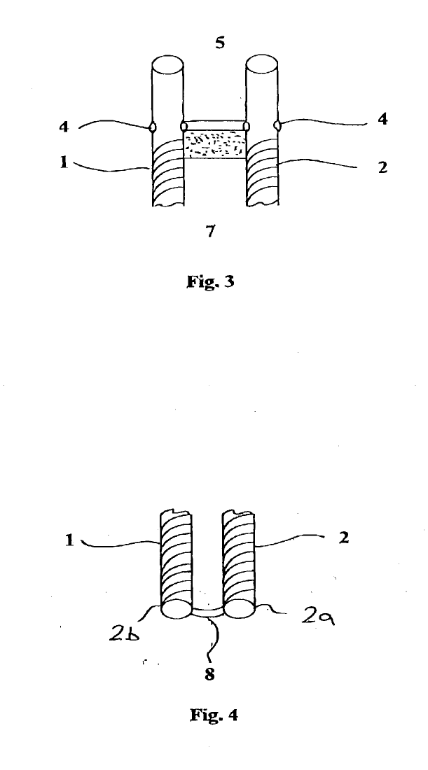

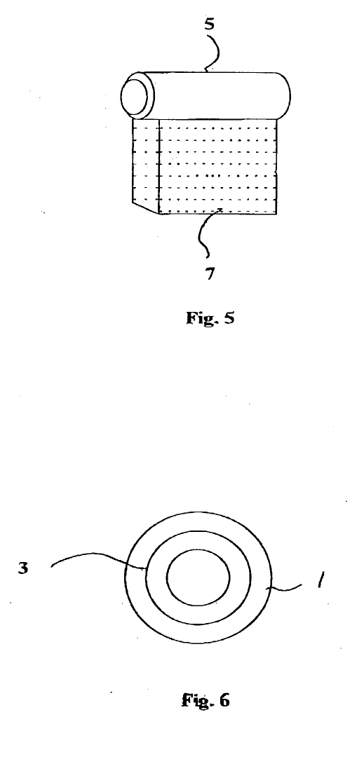

FIGS. 3 to 5 illustrate features of the apparatus shown in FIG. 1;

FIG. 6 is a diagrammatic cross-section taken through a choanal tube which forms part of the apparatus shown in FIG. 1; and

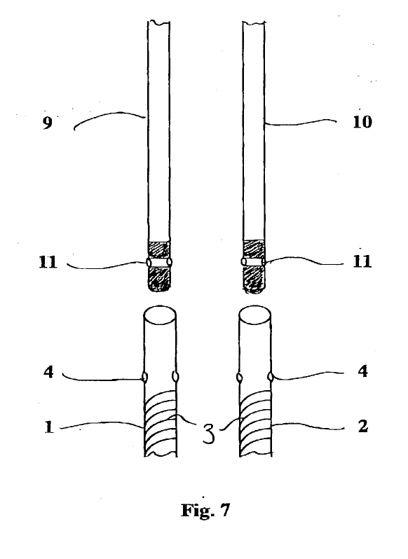

FIG. 7 illustrates additional features of the apparatus of FIG. 1.

The apparatus illustrated in the drawings comprises two side-by-side choanal tubes 1, 2 of a Z79-IT plastics material tested and approved for use in humans. This material may comprise silicone rubber or polyvinylchloride. Embedded wholly and internally within the walls of each choanal tube is a lengthwise extending spiral coil 3 of relatively thin gauge. The material of the coil may be, for example, stainless steel wire. The coils 3 may alternatively be produced from a non-metallic material having the requisite physical properties. The function of the coils is to reinforce the tubes without inhibiting their ability to flex.

Each metallic coil 3 extends over the major part of the length of each choanal tube 1, 2 between a position close to one end 1a, 2a of each tube and a position spaced from the other end 1b, 2b of each tube. The choanal tube ends 1a, 2a are attached to one another by a strip 8 of a Z79-IT plastics material tested and approved for use in humans. The strip 8 is typically arcuate in cross-section with its ends extending into and releasably retained within the ends 1a, 2a of the choanal tubes. Alternatively, the strip ends may be bonded to the external or internal surfaces of the ends 1a, 2a of the choanal tubes. Other means of attaching the strip to the tubes may be employed.

A hollow bridge 5 of plastics material is positioned between the inner surfaces of the end portions 1b, 2b of the choanal tubes. The bridge 5 is held in place by a thread 6 which passes through holes 4 formed in the tubes 1, 2, into and through the hollow bridge 5 and through the tube end portions to positions externally of the tube ends where the thread ends can be tied together. When tied, the choanal tubes lie side-by-side and generally parallel. Attached to the side of the bridge 5 opposite the strip 8 is a rectangular piece of a sponge-like material 7.

As will be seen more clearly from FIG. 6, the coils 3 are wholly embedded within the annular walls of the choanal tubes 1, 2 with no part of the coils exposed. Only one choanal tube and coil are illustrated in FIG. 6.

Typically, the choanal tubes are produced in a range of diameters to correspond with the more likely diameters of surgically created openings formed in tissue causing obstructions in nasal passageways. Thus, a range of choanal tube diameters from 2.5 to 4 mm may be provided.

In the event of bilateral choanal atresia in an infant, airway passageways are surgically formed in the inhibiting tissue. One choanal tube is then introduced into and through one nostril with the plastics strip 8 resting behind the cartilage which separates the two nasal passageways and the tube resting snugly within the nostril. The other choanal tube is then introduced into and through the other nostril and defines a snug fit within the nostril. The protruding tube ends 1b, 2b are then tied together by the thread 6 thereby preventing accidental removal of the tubes from the infant=s nostrils. The piece of sponge 7 is provided to protect the columella of the infant.

To assist placement of the tubes in their final positions, two catheters 9 and 10 having solid tips 9a, 10a are provided. These are illustrated in FIG. 7. The solid tips each contain a through passageway 11 positioned so that the solid tips of the catheters fit snugly inside the ends 1b, 2b of the choanal tubes with each passageway 11 opposite the holes 4 formed in the tubes.

It will be appreciated that the foregoing is exemplary of apparatus In accordance with the invention and that modifications can readily be made thereto without departing from the true scope of the invention as set out in the appended claims.

1. Apparatus for use in the prevention of stenosis following choanal atresia repair in infants, the apparatus comprising two choanal tubes adapted to lie side-by-side each of a plastics material approved for use in humans and each having a lengthwise extending flexible metallic insert embedded internally within its wall(s), a strip of plastics material whose ends are attached one to one end of each of the choanal tubes, and a bridge of plastics material extending between and removably attached to the choanal tubes at or towards their ends remote from the strip of plastics material, the arrangement being such that, in use, the choanal tubes are positioned in the nasal passageways and transnasal openings of an infant with the plastics strip extending behind the cartilage which separates the nasal passageways, and with the bridge positioned adjacent the columella of the infant.

2. Apparatus as claimed in claim 1 wherein each metallic insert comprises a spiral of a metallic material embedded within the respective choanal tube.

3. Apparatus as claimed in claim 1 wherein each metallic insert comprises a spiral of stainless steel embedded within the respective choanal tube.

4. Apparatus as claimed in claim 1 wherein each metallic insert comprises a stainless steel wire coil embedded internally within the respective choanal tube.

5. Apparatus as claimed in claim 1 wherein each choanal tube is produced from silicone rubber.

6. Apparatus as claimed in claim 1 wherein each choanal tube is produced from polyvinylchloride.

7. Apparatus as claimed in claim 1 wherein each metallic insert extends over the major part of the length of the respective choanal tube.

8. Apparatus as claimed in claim 1 wherein the ends of the plastics strip are permanently attached to the ends of the choanal tubes.

9. Apparatus as claimed in claim 1 wherein the ends of the plastics strip are releasably attached to the ends of the choanal tubes.

10. Apparatus as claimed in claim 1 wherein the bridge is held in place by a thread which passes through holes formed in the choanal tubes.

11. Apparatus as claimed in claim 1 wherein a piece of resilient material is attached to that side of the bridge opposite the plastics strip.

12. Apparatus as claimed in claim 11 wherein the resilient material is produced from a sponge-like material.

13. Apparatus as claimed in claim 1 wherein the diameter of each choanal tube lies in the range 2.5 to 4.0 mm.

14. Apparatus for use in the treatment of choanal atresia which comprises two side-by-side choanal tubes each of a material approved for use in humans and each reinforced with an embedded lengthwise extending filament coil, and a strip of material approved for use in humans whose ends are attached to one end of each tube.

15. Apparatus as claimed in claim 14 wherein the material of the choanal tubes and the strip is plastics.

16. Apparatus as claimed in claim 14 in which a bridge of plastics material is releasably secured to each choanal tube at positions at or towards the ends of the tubes remote from the strip of plastics material.

17. Apparatus as claimed in claim 14 wherein the embedded filament coil comprises a spiral of a metallic material.

18. Apparatus as claimed in any one of claim 14 wherein the embedded filament coil comprises stainless steel.

19. Apparatus as claimed in claim 14 wherein the plastics material comprises polyvinylchloride.

20. Apparatus as claimed in claim 14 wherein the plastics strip is permanently attached to the ends of the two choanal tubes.

21. Apparatus as claimed in claim 14 wherein the plastics strip is detachably secured to the ends of the two choanal tubes.
